(19) United States
(12) Patent Application Publication
(10) Pub. No.: US 2013/0123634 A1
(43) Pub. Date: May 16, 2013

(54) SYSTEMS AND METHODS FOR PROMOTING FLOW OF AN ACOUSTICALLY-FAVORABLE MEDIUM OVER A TRANSDUCER OF AN ULTRASOUND IMAGING SYSTEM

(71) Applicant: Boston Scientific SciMed, Inc., Maple Grove, MN (US)

(72) Inventor: Michael J. Tierney, Pleasanton, CA (US)

(73) Assignee: Boston Scientific SciMed, Inc., Maple Grove, MN (US)

(21) Appl. No.: 13/678,085

(22) Filed: Nov. 15, 2012

Related U.S. Application Data
(60) Provisional application No. 61/560,115, filed on Nov. 15, 2011.

(51) Int. Cl. A61B 8/00; A61B 8/12

(52) U.S. Cl. CPC A61B 8/4281; A61B 8/12; A61B 8/4461; A61B 8/4488
USPC 600/444; 600/445

(57) ABSTRACT

A catheter assembly includes an imaging core insertable into a lumen of a sheath of a catheter. The imaging core includes a rotatable driveshaft and a transducer that is coupled to the driveshaft and that rotates with the driveshaft. One or more inlet ports and one or more outlet ports are defined in a distal portion of the sheath. A flow-inducing element is coupled to the driveshaft and rotates with the driveshaft. The flow-inducing element draws fluid into the lumen through the inlet port(s) from the environment external to the sheath and pushes the drawn fluid out of the lumen through the outlet port(s). One of the one or more inlet ports or the one or more outlet ports is disposed proximal to the flow-inducing element and the transducer, and the other of the inlet port(s) or the outlet port(s) is disposed distal to the flow-inducing element and the transducer.

### CROSS-REFERENCE TO RELATED APPLICATIONS

This application claims the benefit under 35 U.S.C. §119(e) of U.S. Provisional Patent Application Ser. No. 61/560,115 filed on Nov. 15, 2011, which is incorporated herein by reference.

### TECHNICAL FIELD

The present invention is directed to the area of ultrasound imaging systems and methods of making and using the systems. The present invention is also directed to an ultrasound imaging system that includes a transducer disposed within a catheter and a flow-inducing element for inducing flow of an acoustically-favorable medium into the catheter and over the transducer, as well as methods of making and using the ultrasound systems, catheter, transducer, and flow-inducing element.

### BACKGROUND

Ultrasound devices insertable into patients have proven diagnostic capabilities for a variety of diseases and disorders. For example, intravascular ultrasound (“IVUS”) imaging systems have been used as an imaging modality for diagnosing blocked blood vessels and providing information to aid medical practitioners in selecting and placing stents and other devices to restore or increase blood flow. IVUS imaging systems have been used to diagnose atheromatous plaque build-up at particular locations within blood vessels. IVUS imaging systems can be used to determine the existence of an intravascular obstruction or stenosis, as well as the nature and degree of the obstruction or stenosis. IVUS imaging systems can be used to visualize segments of a vascular system that may be difficult to visualize using other intravascular imaging techniques, such as angiography, due to, for example, movement (e.g., a beating heart) or obstruction by one or more structures (e.g., one or more blood vessels not desired to be imaged). IVUS imaging systems can be used to monitor or assess ongoing intravascular treatments, such as angiography and stent placement in real (or almost real) time. Moreover, IVUS imaging systems can be used to monitor one or more heart chambers.

IVUS imaging systems have been developed to provide a diagnostic tool for visualizing a variety is diseases or disorders. An IVUS imaging system can include a control module (with a pulse generator, an image processor, and a monitor), a catheter, and one or more transducers disposed in the catheter. The transducer-containing catheter can be positioned in a lumen or cavity within, or in proximity to, a region to be imaged, such as a blood vessel wall or patient tissue in proximity to a blood vessel wall. The pulse generator in the control module generates electrical signals that are delivered to the one or more transducers and transformed to acoustic signals that are transmitted through patient tissue. Reflected signals of the transmitted acoustic signals are absorbed by the one or more transducers and transformed to electric signals. The transformed electric signals are delivered to the image processor and converted to an image displayable on the monitor.

Intracardiac echocardiography (“ICE”) is another ultrasound imaging technique with proven capabilities for use in diagnosing intravascular diseases and disorders. ICE uses acoustic signals to image patient tissue. Acoustic signals emitted from an ICE imager disposed in a catheter are reflected from patient tissue and collected and processed by a coupled ICE control module to form an image. ICE imaging systems can be used to image tissue within a heart chamber.

### BRIEF SUMMARY

In one embodiment, a catheter assembly for an ultrasound system includes an elongated catheter configured and arranged for insertion into the cardiovascular system of a patient. The catheter has a distal end, a proximal end, and a longitudinal length. The catheter includes a sheath with a proximal portion and a distal portion. The sheath defines a lumen extending along the sheath from the proximal portion to the distal portion. An imaging core is configured and arranged for inserting into the lumen of the catheter. The imaging core includes an elongated, rotatable driveshaft having a proximal end and a distal end. The imaging core also includes an imaging device coupled to the distal end of the driveshaft such that rotation of the driveshaft causes a corresponding rotation of the imaging device. The imaging device includes at least one transducer mounted to the imaging device. The at least one transducer is configured and arranged for transforming applied electrical signals to acoustic signals and also for transforming received echo signals to electrical signals. At least one inlet port is defined in the distal portion of the sheath. The at least one inlet port extends between the lumen and the environment external to the catheter. At least one outlet port is defined in the distal portion of the sheath. The at least one outlet port extends between the lumen and the environment external to the catheter. A flow-inducing element is coupled to the driveshaft such that rotation of the driveshaft causes a corresponding rotation of the flow-inducing element. The flow-inducing element is configured and arranged to draw fluid into the lumen through the at least one inlet port from the environment external to the sheath and push the drawn fluid out of the lumen through the at least one outlet port to the environment external to the sheath. One of the at least one inlet port or the at least one outlet port is disposed proximal to the flow-inducing element and the at least one transducer, and the other of the at least one inlet port or the at least one outlet port is disposed distal to the flow-inducing element and the at least one transducer.

In another embodiment, a catheter assembly for an ultrasound system includes an elongated catheter configured and arranged for insertion into the cardiovascular system of a patient. The catheter has a distal end with a first diameter, a proximal end, and a longitudinal length. The catheter includes a sheath with a proximal portion and a distal portion. The sheath defines a lumen extending along the sheath from the proximal portion to the distal portion. The catheter assembly also includes an imaging core with a longitudinal length that is substantially less than the longitudinal length of the catheter. The imaging core is configured and arranged for inserting into the lumen of the catheter and disposing at the distal end of the catheter. The imaging core includes a rotatable driveshaft having a proximal end and a distal end, and an imaging device coupled to the distal end of the driveshaft. The imaging device includes at least one transducer mounted to the imaging device such that rotation of the driveshaft causes corresponding rotation of the imaging device. The at least one transducer is configured and arranged for transforming applied electrical signals to acoustic signals and also for transforming received echo signals to electrical signals. The imaging core further includes a transformer disposed at the proximal end of the driveshaft; at least one imaging core conductor coupling the at least one transducer to the transformer; and a motor having a diameter. The motor is coupled to the driveshaft between the at least one transducer and the transformer and is configured and arranged to rotate the driveshaft. The motor includes a magnet driven to rotate by at least two magnetic field windings. At least one inlet port is defined in the distal portion of the sheath and extends between the lumen and the environment external to the catheter. At least one outlet port is defined in the distal portion of the sheath and extends between the lumen and the environment external to the catheter. A flow-inducing element is coupled to the driveshaft such that rotation of the driveshaft causes a corresponding rotation of the flow-inducing element. The flow-inducing element is configured and arranged to draw fluid into the lumen through the at least one inlet port from the environment external to the sheath and push the drawn fluid out of the lumen through the at least one outlet port to the environment external to the sheath. One of the at least one inlet port or the at least one outlet port is disposed proximal to the flow-inducing element and the at least one transducer, and the other of the at least one inlet port or the at least one outlet port is disposed distal to the flow-inducing element and the at least one transducer.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an intravascular ultrasound imaging system, according to the invention;

FIG. 2 is a schematic side view of one embodiment of a catheter of an intravascular ultrasound imaging system, according to the invention;

FIG. 3 is a schematic perspective view of one embodiment of a distal end of the catheter shown in FIG. 2 with an imaging core disposed in a lumen defined in the catheter, according to the invention;

FIG. 4 is a schematic longitudinal cross-sectional view of another embodiment of a distal end of a catheter, the distal end of the catheter including an imaging core with a rotatable magnet, a transformer, and one or more rotating transducers, according to the invention;

FIG. 5A is a schematic longitudinal cross-sectional view of one embodiment of the distal end of the catheter of FIG. 3 disposed in a blood vessel with blood flow in a first direction, the catheter including a flow-inducing element for drawing blood from the blood vessel into the catheter, according to the invention;

FIG. 5B is a schematic longitudinal cross-sectional view of one embodiment of the distal end of the catheter of FIG. 3 disposed in a blood vessel with blood flow in a second direction, the catheter including a flow-inducing element for drawing blood from the blood vessel into the catheter, according to the invention;

FIG. 6A a schematic longitudinal cross-section view of a distal end of catheter of FIG. 4 disposed in a blood vessel with blood flow in a first direction, the catheter including a flow-inducing element for drawing blood from the blood vessel into the catheter, according to the invention; and

FIG. 6B a schematic longitudinal cross-section view of a distal end of catheter of FIG. 4 disposed in a blood vessel with blood flow in a second direction, the catheter including a flow-inducing element for drawing blood from the blood vessel into the catheter, according to the invention.

### DETAILED DESCRIPTION

The present invention is directed to the area of ultrasound imaging systems and methods of making and using the systems. The present invention is also directed to an ultrasound imaging system that includes a transducer disposed within a catheter and a flow-inducing element for inducing flow of an acoustically-favorable medium into the catheter and over the transducer, as well as methods of making and using the ultrasound systems, catheter, transducer, and flow-inducing element.

Suitable ultrasound imaging systems utilizing catheters include, for example, intravascular ultrasound (“IVUS”) and intracardiac echocardiography (“ICE”) systems. These systems may include one or more transducers disposed on a distal end of a catheter configured and arranged for percutaneous insertion into a patient. Examples of IVUS imaging systems with catheters are found in, for example, U.S. Pat. Nos. 6,945,938; 7,246,959; and 7,306,561; as well as U.S. Patent Application Publication Nos. 2006/0100522; 2006/0106320; 2006/0173350; 2006/0253028; 2007/0016054; and 2007/0038111; all of which are incorporated herein by reference.

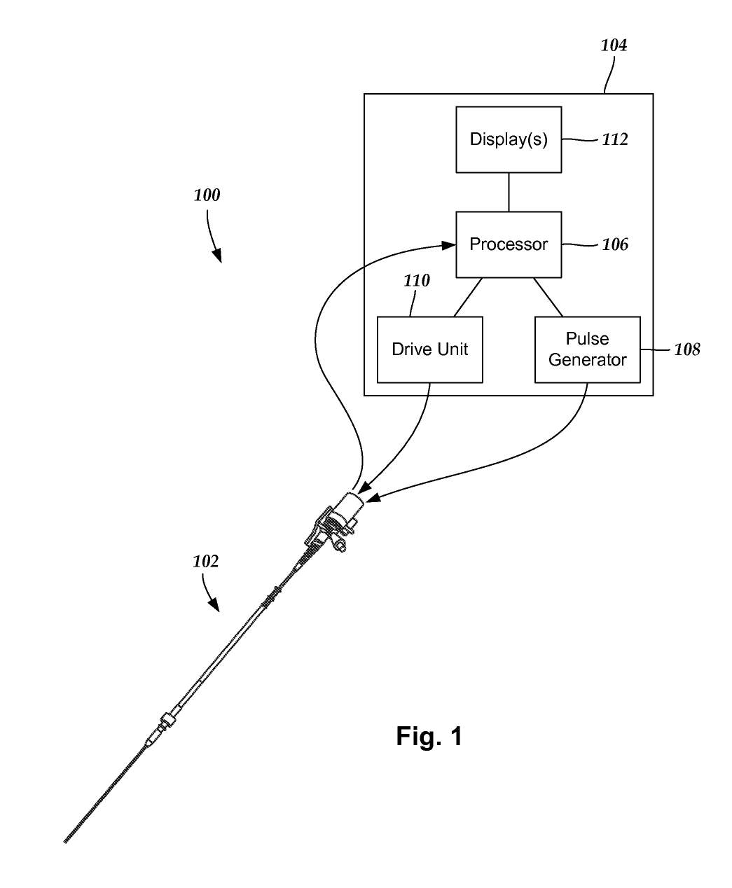

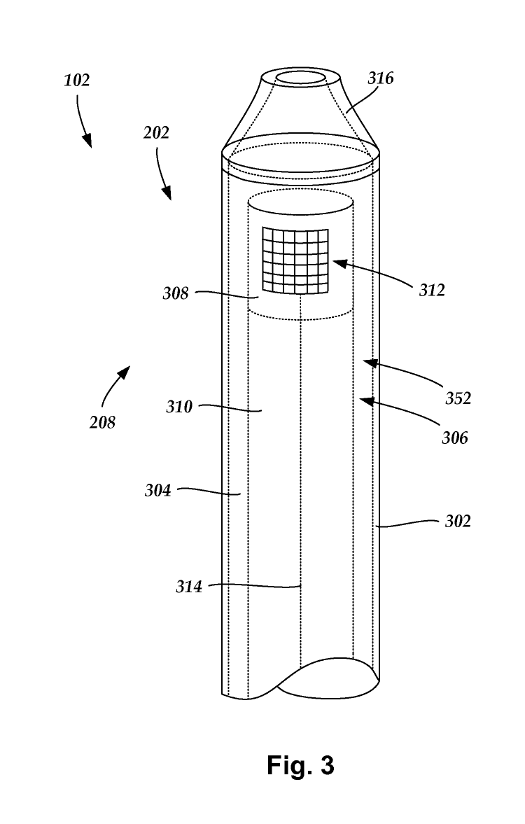

FIG. 1 illustrates schematically one embodiment of an IVUS imaging system 100. An ICE imaging system is similar. The IVUS imaging system 100 includes a catheter 102 that is coupleable to a control module 104. The control module 104 may include, for example, a processor 106, a pulse generator 108, a drive unit 110, and one or more displays 112. In at least some embodiments, the pulse generator 108 forms electric signals that may be input to one or more transducers (312 in FIG. 3) disposed in the catheter 102. In at least some embodiments, mechanical energy from the drive unit 110 may be used to drive an imaging core (306 in FIG. 3) disposed in the catheter 102.

In at least some embodiments, electrical signals transmitted from the one or more transducers (312 in FIG. 3) may be input to the processor 106 for processing. In at least some embodiments, the processed electrical signals from the one or more transducers (312 in FIG. 3) may be displayed as one or more images on the one or more displays 112. In at least some embodiments, the processor 106 may also be used to control the functioning of one or more of the other components of the control module 104. For example, the processor 106 may be used to control at least one of the frequency or duration of the electrical signals transmitted from the pulse generator 108, the rotation rate of the imaging core (306 in FIG. 3) by the drive unit 110, the velocity or length of the pullback of the imaging core (306 in FIG. 3) by the drive unit 110, or one or more properties of one or more images formed on the one or more displays 112.

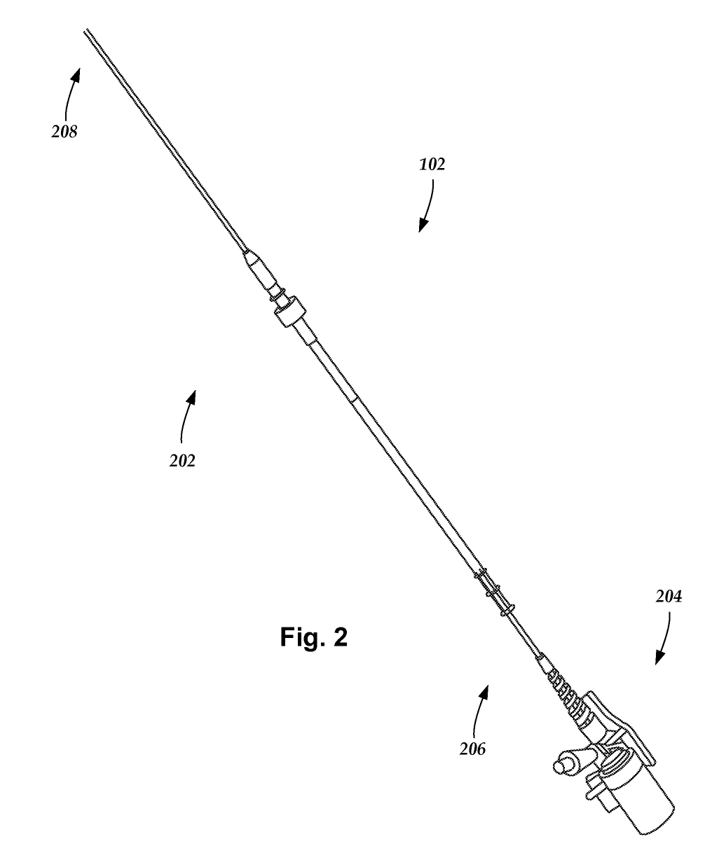

FIG. 2 is a schematic side view of one embodiment of the catheter 102 of the IVUS imaging system (100 in FIG. 1). The catheter 102 includes an elongated member 202 and a hub 204. The elongated member 202 includes a proximal end 206 and a distal end 208. In FIG. 2, the proximal end 206 of the elongated member 202 is coupled to the catheter hub 204 and the distal end 208 of the elongated member is configured and arranged for percutaneous insertion into a patient. In some embodiments, the elongated member 202 and the hub 204 are formed as a unitary body. In other embodiments, the elongated member 202 and the catheter hub 204 are formed separately and subsequently assembled together.

FIG. 3 is a schematic perspective view of one embodiment of the distal end 208 of the catheter 102. The catheter 102 includes a sheath 302 having a distal portion 352 and a proximal portion (not shown). The sheath 302 defines a lumen 304 extending from the distal portion 352 of the sheath 302 to the distal portion. An imaging core 306 is disposed in the lumen 304. The imaging core 306 includes an imaging device 308 coupled to a distal end of a driveshaft 310.

The sheath 302 may be formed from any flexible, biocompatible material suitable for insertion into a patient. Examples of suitable materials include, for example, polyethylene, polyurethane, plastic, spiral-cut stainless steel, nitinol hypotube, and the like or combinations thereof.

One or more transducers 312 may be mounted to the imaging device 308 and employed to transmit and receive acoustic signals. In a preferred embodiment (as shown in FIG. 3), an array of transducers 312 are mounted to the imaging device 308. In other embodiments, a single transducer may be employed. In at least some embodiments, multiple transducers in an irregular-array may be employed. Any number of transducers 312 can be used. For example, there can be one, two, three, four, five, six, seven, eight, nine, ten, twelve, fifteen, sixteen, twenty, twenty-five, fifty, one hundred, five hundred, one thousand, or more transducers. As will be recognized, other numbers of transducers may also be used.

The one or more transducers 312 may be formed from one or more known materials capable of transforming applied electrical signals to pressure distortions on the surface of the one or more transducers 312, and vice versa. Examples of suitable materials include piezoelectric ceramic materials, piezocomposite materials, piezoelectric plastics, barium titanates, lead zirconate titanates, lead metaniobates, polyvinylidenefluorides, and the like.

The pressure distortions on the surface of the one or more transducers 312 form acoustic signals of a frequency based on the resonant frequencies of the one or more transducers 312. The resonant frequencies of the one or more transducers 312 may be affected by the size, shape, and material used to form the one or more transducers 312. The one or more transducers 312 may be formed in any shape suitable for positioning within the catheter 102 and for propagating acoustic signals of a desired frequency in one or more selected directions. For example, transducers may be disc-shaped, block-shaped, rectangular-shaped, oval-shaped, and the like. The one or more transducers may be formed in the desired shape by any process including, for example, dicing, dice and fill, machining, micro fabrication, and the like.

As an example, each of the one or more transducers 312 may include a layer of piezoelectric material sandwiched between a conductive acoustic lens and a conductive backing material formed from an acoustically absorbent material (e.g., an epoxy substrate with tungsten particles). During operation, the piezoelectric layer may be electrically excited by both the backing material and the acoustic lens to cause the emission of acoustic signals.

In at least some embodiments, the one or more transducers 312 can be used to form a radial cross-sectional image of a surrounding space. Thus, for example, when the one or more transducers 312 are disposed in the catheter 102 and inserted into a blood vessel of a patient, the one more transducers 312 may be used to form a composite image of the walls of the blood vessel and tissue surrounding the blood vessel by stitching together a plurality of individual image frames.

In at least some embodiments, the imaging core 306 may be rotated about a longitudinal axis of the catheter 102 while being disposed in the distal portion 352 of the sheath 302. As the imaging core 306 rotates, the one or more transducers 312 emit acoustic signal in different radial directions. When an emitted acoustic signal with sufficient energy encounters one or more medium boundaries, such as one or more tissue boundaries, a portion of the emitted acoustic signal is reflected back to the emitting transducer as an echo signal. Each echo signal that reaches a transducer with sufficient energy to be detected is transformed to an electrical signal in the receiving transducer. The one or more transformed electrical signals are transmitted to the control module (104 in FIG. 1) where the processor 106 processes the electrical-signal characteristics to generate a displayable image frame of the imaged region based, at least in part, on a collection of information from each of the acoustic signals transmitted and the echo signals received. In at least some embodiments, the rotation of the one or more transducers 312 is driven by the drive unit 110 disposed in the control module (104 in FIG. 1), via the driveshaft 310 extending along the sheath 302 of the catheter 102.

As the one or more transducers 312 rotate about the longitudinal axis of the catheter 102 emitting acoustic signals, a plurality of image frames are formed that collectively form a composite radial cross-sectional image of a portion of the region surrounding the one or more transducers 312, such as the walls of a blood vessel of interest and the tissue surrounding the blood vessel. In at least some embodiments, one or more of the image frames can be displayed on the one or more displays 112. In at least some embodiments, the radial cross-sectional composite image can be displayed on the one or more displays 112.

In at least some embodiments, the imaging core 306 may also move longitudinally (i.e., translate) along the blood vessel within which the catheter 102 is inserted so that a plurality of composite cross-sectional images may be formed into one or more larger composite images that include an axial length of the blood vessel. In at least some embodiments, during an imaging procedure the one or more transducers 312 may be retracted (i.e., pulled back) along the longitudinal length of the catheter 102. In at least some embodiments, the catheter 102 includes at least one section that can be retracted during pullback of the one or more transducers 312. In at least some embodiments, the drive unit 110 drives the pullback of the imaging core 306 within the catheter 102. In at least some embodiments, the drive unit 110 pullback distance of the imaging core is at least 5 cm, 10 cm, 15 cm, 20 cm, 25 cm or more. In at least some embodiments, the catheter 102 pullback occurs along one or more telescoping sections.

The quality of imaging at different depths from the one or more transducers 312 may be affected by one or more factors including, for example, bandwidth, transducer focus, beam pattern, as well as the frequency of the acoustic signal. The frequency of the acoustic signal output from the one or more transducers 312 may also affect the penetration depth of the acoustic signal output from the one or more transducers 312. In general, as the frequency of an acoustic signal is lowered, the depth of the penetration of the acoustic signal within patient tissue increases. In at least some embodiments, the IVUS imaging system 100 operates within a frequency range of 5 MHz to 60 MHz.

In at least some embodiments, one or more transducer conductors 314 electrically couple the transducers 312 to the control module 104 (See FIG. 1). In at least some embodiments, the one or more transducer conductors 314 extend along the driveshaft 310.

In at least some embodiments, the imaging device 308 may be inserted in the lumen of the catheter 102. In at least some embodiments, the catheter 102 (and imaging device 308) may be inserted percutaneously into a patient via an accessible blood vessel, such as the femoral artery or vein, at a site remote from a target imaging location. The catheter 102 may then be advanced through patient vasculature to the target imaging location, such as a portion of a selected blood vessel (e.g., a peripheral blood vessel, a coronary blood vessel, or other blood vessel), or one or more chambers of the patient's heart.

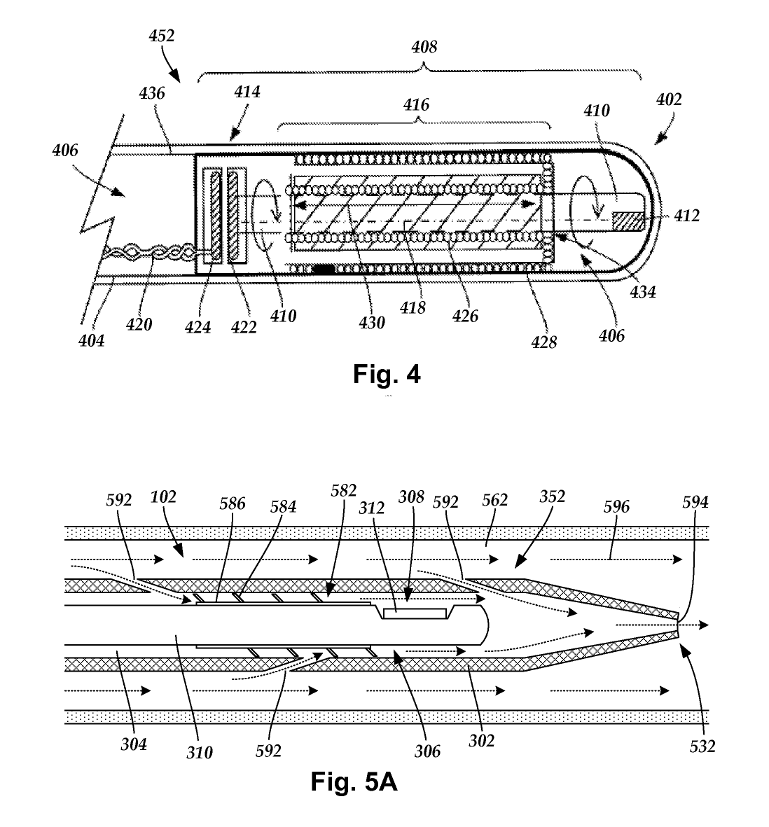

Turning to FIG. 4, in other embodiments an imaging core includes a motor that is at least partially disposed in the imaging core. In at least some embodiments, the motor is magnetic. The motor may include a rotor and a stator. In at least some embodiments, the rotor is a rotatable magnet and the stator includes a plurality of magnetic field windings configured and arranged to rotate the magnet by a generated magnetic field. Examples of IVUS or ICE imaging systems that include imaging cores at least partially disposed in catheters, and magnetic motors that rotate either the imaging devices, or mirrors that are disposed in the imaging cores and that are in proximity to the imaging devices, are found in, for example, U.S. Pat. Nos. 8,298,149; U.S. Patent Application Publication Nos. 2010/0249599; 2010/0249604; 2011/0071400; and 2011/0071401; U.S. Application Ser. Nos. 61/286,674; and 61/288,719, all of which are incorporated herein by reference.

The magnetic field windings (“windings”) may be disposed in the imaging core or, alternately, may be disposed external to the imaging core. In at least some embodiments, the windings may be disposed external to the catheter, or even external to a patient during an imaging procedure. In at least some embodiments, the imaging core is configured and arranged for insertion into the lumen of the catheter.

In at least some embodiments, the imaging core is configured and arranged such that rotation of the magnet causes a corresponding rotation of the one or more transducers configured and arranged to transmit energy to patient tissue and receive corresponding echo signals, as described above with reference to FIG. 3. In alternate embodiments, the one or more transducers do not rotate. Instead, the imaging core is configured and arranged such that rotation of the magnet causes a corresponding rotation of a tilted mirror configured and arranged to redirect energy between the one or more fixed transducers and patient tissue.

FIG. 4 is a schematic longitudinal cross-sectional view of one embodiment of a distal end 452 of a catheter 402. The catheter 402 includes a sheath 404 and a lumen 406. In FIG. 4, the imaging core 408 is shown disposed in the lumen 406 of the sheath 404 at a distal portion 452 of the sheath 404. The imaging core 408 includes a rotatable driveshaft 410 with one or more transducers 412 coupled to a distal end of the driveshaft 410, and a transformer 414 coupled to a proximal end of the driveshaft 410. The imaging core 408 also includes a motor 416 coupled to the driveshaft 410. One or more imaging core conductors 418 electrically couple the one or more transducers 412 to the transformer 414. In at least some embodiments, the one or more imaging core conductors 418 extend within the driveshaft 410. One or more catheter conductors 420 electrically couple the transformer 414 to the control module (104 in FIG. 1). In at least some embodiments, the one or more of the catheter conductors 420 may extend along at least a portion of a length of the catheter 402 as shielded electrical cables, such as a coaxial cable, or a twisted pair cable, or the like. In at least some embodiments, the one or more catheter conductors 420 extend through the sheath 404.

In FIG. 4, the transformer 414 is shown disposed on the imaging core 408. In at least some embodiments, the transformer 414 includes a rotating component 422 coupled to the driveshaft 410 and a stationary component 424 disposed spaced apart from the rotating component 414. In some embodiments, the stationary part 424 is proximal to, and immediately adjacent to, the rotating component 422. The rotating component 422 is electrically coupled to the one or more transducers 412 via the one or more imaging core conductors 418 disposed in the imaging core 408. The stationary component 416 is electrically coupled to the control module (104 in FIG. 1) via one or more conductors 420 disposed in the lumen 406. Current is inductively passed between the rotating component 422 and the stationary component 424 (e.g., a rotor and a stator, or a rotating pancake coil and a stationary pancake coil, or the like).

In at least some embodiments, the transformer 414 is positioned at a proximal end of the imaging core 408. In at least some embodiments, the components 422 and 424 of the transformer 414 are disposed in a ferrite form. In at least some embodiments, the components 422 and 424 are smaller in size than components conventionally positioned at the proximal end of the catheter.

In FIG. 4, the motor 416 includes a magnet 426 and windings 428 both disposed in the imaging core 408. In at least some embodiments, the magnet 426 is a permanent magnet with a longitudinal axis, indicated by a two-headed arrow 430, which is coaxial with the longitudinal axes of each of the imaging core 408 and the driveshaft 410.

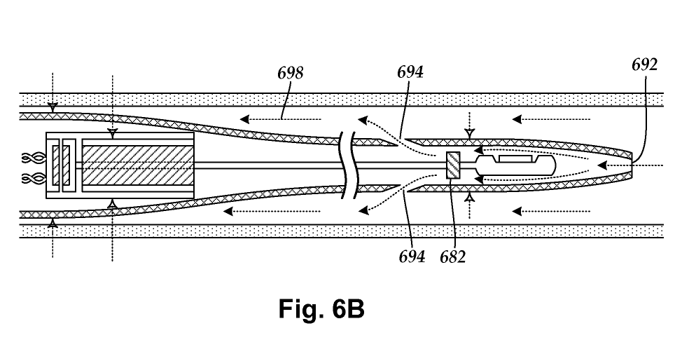

In at least some embodiments, the magnet 426 is coupled to the driveshaft 410 and is configured and arranged to rotate the driveshaft 410 during operation. In at least some embodiments, the magnet 426 defines an aperture 434 along the longitudinal axis 430 of the magnet 426. In at least some embodiments, the driveshaft 410 and the one or more imaging core conductors 418 extend through the aperture 434. In at least some other embodiments, the drive shaft 410 is discontinuous and, for example, couples to the magnet 426 at opposing ends of the magnet 426. In which case, the one or more imaging core conductors 418 still extend through the aperture 434. In at least some embodiments, the magnet 426 is coupled to the driveshaft 410 by an adhesive. Alternatively, in some embodiments the driveshaft 410 and the magnet 426 can be machined from a single block of magnetic material with the aperture 434 drilled down a length of the driveshaft 410 for receiving the imaging core conductors 418. The windings 428 are provided with power from the control module (104 in FIG. 1) via one or more motor conductors 436. In at least some embodiments, the one or more motor conductors 436 extend through the sheath 404. In alternate embodiments, the one or more motor conductors 436 extend along the lumen 406 (see e.g., FIG. 6).

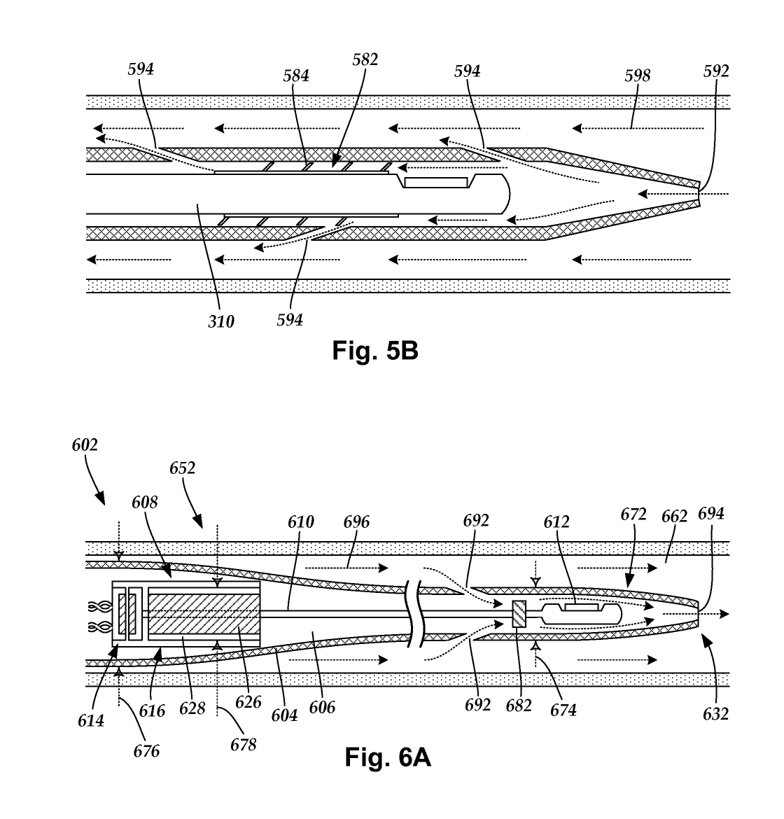

Turning to FIGS. 5A-5B, acoustic signals propagating from the one or more transducers 312, 412 propagate through a portion of the lumen 304, 406 surrounding the imaging device 308, 408 before passing through the sheath 302, 404 to the region exterior of the catheter 102, 402 such as a blood vessel or a chamber of a heart. Likewise, echo signals reflected back to the one or more transducers 312, 412 from medium boundaries also propagate through a portion of the lumen 304, 406. Typically, air is not a desirable transmission medium and image quality may, consequently, be reduced when acoustic signals or echo signals are required by catheter design to propagate through air. In the MHz range, acoustic signals may not propagate at all through air. Accordingly, it is typically advantageous, and in some cases necessary, to purge air from the lumen 304, 406 surrounding the one or more transducers 312, 412 prior to (or one or more times during) the performance of an imaging procedure.

One technique for purging air surrounding the one or more transducers 312, 412 is to flush the lumen 304, 406 with an acoustically-favorable medium through which acoustic signals more easily propagate than through air Acoustically-favorable media may include one or more solvents such as, for example, water. An acoustically-favorable medium may include one or more solutes mixed with the one or more solvents such as, for example, one or more salts. Blood may be an acoustically-favorable medium. In at least some embodiments, one or more agents may also be added, for example, to decrease the potential advancement of corrosion or microbial growth. In at least some embodiments, an acoustically-favorable medium may include a gel, and the like.

When using a conventional IVUS imaging system, a lumen of a catheter may be manually flushed to remove air at the beginning of an IVUS imaging procedure. Additionally, the lumen of the catheter may also be manually flushed of air one or more additional times during the course of the IVUS imaging procedure. Unfortunately, each manual flushing of air from the catheter lumen can add to the amount of time it takes to perform an IVUS imaging procedure on a patient.

As herein described, a rotatable flow-inducing element can be used to induce flow of an acoustically-favorable medium along a portion of the lumen within which the transducer is disposed. The flow of acoustically-favorable medium induced by the flow-inducing element may remove the need for a medical practitioner to manually flush air from the lumen before or during an imaging procedure. The flow-inducing element may be used either with imaging cores that do not include motors (see e.g., FIG. 3), or imaging cores that do include motors (see e.g., FIG. 4). In at least some embodiments, the flow-inducing element is coupled to the same component that rotates the transducer (e.g., the driveshaft). In which case, the flow-inducing element may rotate with the transducer. In embodiments that include a stationary transducer and a rotating mirror, the flow-inducing element may be coupled to the same component that rotates the mirror (e.g., the driveshaft). In at least some embodiments, the flow-inducing element includes a screw pump, or Archimedes's screw. In at least some embodiments, the flow-inducing element includes an impellor.

The flow-inducing element is configured and arranged to move or guide fluid from the environment external to the catheter within which the flow-inducing element is disposed into the lumen. For example, when the catheter is disposed in a blood vessel, the flow-inducing element is configured and arranged to push fluid from inside the lumen of the catheter out to the blood vessel through one or more inlet ports. The pushing of the fluid from the lumen causes a negative pressure to be formed in the lumen, which causes blood to be drawn into the lumen from the blood vessel through one or more inlet ports. In at least some embodiments, the flow-inducing element and the one or more transducers are disposed between at least one of the inlet ports on one end and at least one of the outlet ports on an opposing end such that the flow-inducing element induces flow across the one or more transducers so that the one or more transducers remain immersed and surrounded by a relatively gas-free environment.

In at least some embodiments the flow-inducing element may be used with an imaging core that does not include a motor (see e.g., FIG. 3). FIG. 5A is a schematic longitudinal cross-sectional view of one embodiment of the distal portion 352 of the sheath 302 of the catheter 102 disposed in a blood vessel 562 with blood flowing in a first direction. FIG. 5B is a schematic longitudinal cross-sectional view of one embodiment of the distal portion 352 of the sheath 302 catheter 102 disposed in the blood vessel 562 with blood flowing in a second direction that is opposite from the first direction. The imaging core 306 is disposed in the lumen 304 of the sheath 302. The imaging core 306 includes the imaging device 308 coupled to a distal end of the driveshaft 310. The imaging device 308 includes one or more transducers 312.

A flow-inducing element 582 is disposed in the catheter 102 and is in fluid communication with the lumen 304 and the imaging device 308. In at least some embodiments, the flow-inducing element 582 is configured and arranged to rotate during an imaging procedure. In at least some embodiments, the flow-inducing element 582 is configured and arranged to rotate whenever the driveshaft 310 rotates.

In at least some embodiments, the flow-inducing element 582 enables air to be flushed from the lumen 304 and replaced by an acoustically-favorable medium disposed in the environment disposed external to (and adjacent to) the catheter 102. Thus, when the catheter 102 is disposed in the blood vessel 562, blood may be pumped through the lumen 304 of the catheter 102. In at least some embodiments, the flow-inducing element 582 enables air to be flushed from the lumen 304 prior to an IVUS imaging procedure. In at least some embodiments, the flow-inducing element 582 enables air to be flushed from the lumen 304 during an IVUS imaging procedure.

In FIGS. 5A-5B, the flow-inducing element 582 is shown disposed on the driveshaft 310. In at least some embodiments, the flow-inducing element 582 is affixed (e.g., using an adhesive, interference fit, or the like) to the driveshaft 310. In at least some embodiments, the flow-inducing element 582 and the driveshaft 310 are inseparable from one another. In at least some embodiments, the flow-inducing element 582 and the driveshaft 310 are formed from a single piece of material (i.e., a unitary structure). In at least some embodiments, the flow-inducing element 582 is fixedly coupled to the driveshaft 310 such that rotation of the driveshaft 310 causes a corresponding rotation of the flow-inducing element 582.

In at least some embodiments, the flow-inducing element 582 includes one or more screw pumps, impellors, or the like. In FIGS. 5A-5B, the flow-inducing element is shown as a screw pump that includes one or more blades 584. In at least some embodiments, the one or more blades 584 are coupled directly to the driveshaft 310. In other embodiments, the one or more blades 584 are coupled to a sleeve 586 that, in turn, is coupled to the driveshaft 310.

The sheath 302 defines one or more inlet ports, such as inlet port 592, and one or more outlet ports 594. The one or more inlet ports 592 and one or more outlet ports 594 enable fluid from the environment external to the sheath 302 to enter and exit, respectively, the lumen 304. In at least some embodiments, the flow-inducing element 582 is configured and arranged to push fluid and gas from the lumen 304 through the one or more outlet ports 594. Pushing the fluid and gas creates a negative pressure within the lumen 304, thereby drawing fluid from the environment external to the sheath 302 into the lumen 304 through the one or more inlet ports 592. The flow-inducing element 582, inlet ports 592, and outlet ports 594 are configured and arranged such that, as fluid is drawn along the lumen 304 the fluid flows over the one or more transducers 312. Thus, for example, when the catheter 102 is disposed in a blood vessel, such as the blood vessel 562, operation of the flow-inducing element 582 causes blood (which is typically an acoustically-favorable medium) to enter into the lumen 304 through at least one of the one or more inlet ports 592, flow along at least a portion of the lumen 304 over the one or more transducers 312, and exit the lumen 304 (along with gas) through the one or more outlet ports 594.

The sheath 302 may define any suitable number of inlet ports 592 including, for example, one, two, three, four, five, six, seven, eight, nine, ten, twenty, thirty, forty, fifty, or more inlet ports 592. The one or more inlet ports 592 can be formed in any suitable shape including, for example, linear, round, oval, triangular, rectangular, or the like or combinations thereof. The one or more inlet ports 592 can be any suitable non-geometric shape including, for example, cruciform, wavy line, or the like or combination s thereof. The one or more inlet ports 592 may also be irregularly-shaped. The one or more inlet ports 592 may be formed in the sheath 302 using any suitable technique (e.g., laser drilling, or the like).

The one or more inlet ports 592 may extend through the sheath 302 at any suitable angle relative to a longitudinal length of the catheter 102. In at least some embodiments, at least one of the one or more inlet ports 592 extends perpendicular to the longitudinal length of the catheter 102. In at least some embodiments, at least one of the one or more inlet ports 592 extends parallel to the longitudinal length of the catheter 102. In at least some embodiments, at least one of the one or more inlet ports 592 extends through the sheath 302 at an angle that is neither perpendicular nor parallel to the longitudinal length of the catheter 102 (e.g., at a slant).

In at least some embodiments, at least one of the one or more inlet ports 592 is slanted such that the inlet port 592 opens into the lumen 304 at a location that is more distal along the catheter 102 than a location where that inlet port 592 opens to an outer surface of the sheath 302. In at least some embodiments, at least one of the one or more inlet ports 592 is slanted such that the inlet port 592 opens into the lumen 304 at a location that is more proximal along the catheter 102 than a location where that inlet port 592 opens to an outer surface of the sheath 302. In at least some embodiments, at least one of the one or more inlet ports 592 are slanted in the direction of overall blood flow within the blood vessel within which the catheter 102 is disposed such that, when the blood enters the lumen 304, the blood is diverted from its direction outside the catheter 102 by no more than ninety, eighty, seventy, sixty, fifty, forty, or thirty degrees. In FIG. 5A, the direction of blood flow within the blood vessel 562 is shown by a plurality of arrows, such as arrow 596.

The one or more inlet ports 592 may be defined at any suitable location along the distal portion 352 of the sheath 302. In at least some embodiments, at least one of the one or more inlet ports 592 is located proximal along the longitudinal length of the catheter 102 to the one or more transducers 312. In at least some embodiments, at least one of the one or more inlet ports 592 is located distal along the longitudinal length of the catheter 102 to the flow-inducing element 582. In embodiments where the driveshaft 310 is longitudinally translatable along the longitudinal length of the catheter 102, it may be advantageous to longitudinally-offset at least one of the inlet ports 592 from at least one of the outlet ports 594 along the longitudinal length of the catheter 102 so that at least one of the inlet ports 592 is located on an opposing side of the flow-inducing element 582 from at least one of the outlet ports 592 regardless of the positioning of the flow-inducing element 582 at the distal portion 352 of the sheath 302. In at least some embodiments, the proximal-most inlet port 592 is defined in the sheath 302 such that when the one or more transducers 312 are at their most proximal position in the lumen 304 during pullback, the proximal-most inlet port 592 is proximal to the one or more transducers 312 by no more than 80 mm, 90 mm, 100 mm, 110 mm, 120 mm, 130 mm, 140 mm, 150 mm. In at least some embodiments, the one or more outlet ports 594 are disposed at a distal-most tip 532 of the catheter 102.

The one or more outlet ports 594 may be disposed at any suitable location along the distal portion 352 of the sheath 302. In FIG. 5A, the one or more outlet ports 594 are located distal to the one or more transducers 312. In at least some embodiments, at least one of the one or more outlet ports 594 is disposed distal to at least one of the one or more inlet ports 592. In at least some embodiments, at least one of the one or more outlet ports 594 is disposed proximal to at least one of the one or more inlet ports 592. In at least some embodiments, each of the one or more outlet ports 594 is disposed distal to each of the one or more inlet ports 592. In at least some embodiments, each of the one or more outlet ports 594 is disposed proximal to each of the one or more inlet ports 592. In at least some embodiments, the one or more inlet ports 592 are disposed at the distal-most tip 532 of the catheter 102.

The sheath 302 may define any suitable number of outlet ports 594 including, for example, one, two, three, four, five, six, seven, eight, nine, ten, twenty, thirty, forty, fifty, or more outlet ports 594. The one or more outlet ports 594 can be formed in any suitable shape including, for example, linear, round, oval, triangular, rectangular, or the like or combinations thereof. The one or more outlet ports 594 can be any suitable non-geometric shape including, for example, cruciform, wavy line, or the like or combination s thereof. The one or more outlet ports 594 may also be irregularly-shaped. The one or more outlet ports 594 may be formed in the sheath 302 using any suitable technique (e.g., laser drilling, or the like).

The one or more outlet ports 594 may extend through the sheath 302 at any suitable angle relative to a longitudinal length of the catheter 102. In at least some embodiments, at least one of the one or more outlet ports 594 extends perpendicular to the longitudinal length of the catheter 102. In at least some embodiments, at least one of the one or more outlet ports 594 extends parallel to the longitudinal length of the catheter 102. In at least some embodiments, at least one of the one or more outlet ports 594 extends through the sheath 302 at an angle that is not parallel to the longitudinal length of the catheter 102 (e.g., at a slant).

In at least some embodiments, at least one of the one or more outlet ports 594 is slanted such that the outlet port 594 opens into the lumen 304 at a location that is more distal along the catheter 102 than a location where that outlet port 594 opens to an outer surface of the sheath 302. In at least some embodiments, at least one of the one or more outlet ports 594 is slanted such that the outlet port 594 opens into the lumen 304 at a location that is more proximal along the catheter 102 than a location where that outlet port 594 opens to an outer surface of the sheath 302.

It may be advantageous to dispose at least one of the one or more inlet ports 592 at a location along the length of the catheter 102 such that, when acoustically-favorable medium (e.g., blood) external to the catheter 102 is drawn into the lumen 304 the acoustically-favorable medium passes over the one or more transducers 312 before exiting through the one or more outlet ports 594, thereby ensuring that the one or more transducers 312 remain immersed in the acoustically-favorable medium, and that the newly drawn acoustically-favorable medium (without gas) replaces the old acoustically-favorable medium (with gas).

It may be advantageous to position the one or more inlet ports 592 relative to the one or more outlet ports 594 such that the acoustically-favorable medium flows within the lumen 304 in the same direction as the overall flow of the acoustically-favorable medium in the blood vessel 562 external to the catheter 102, as shown by arrows 596 in FIG. 5A. Thus, in instances where the overall direction of blood flow within the blood vessel 562 is opposite from the directions indicated by the arrows 596 in FIG. 5A, it may be advantageous to reverse the positioning of the one or more inlet ports 592, and the one or more outlet ports 594.

FIG. 5B shows blood flowing in a direction indicated by arrows, such as arrow 598. The arrows 598 indicate a direction that is opposite from the direction shown by arrows 596 in FIG. 5A. In FIG. 5B, the one or more inlet ports 592 and the one or more outlet ports 594 are positioned in opposite locations from the positions shown in FIG. 5A. In some cases, the directionality of the negative pressure created by the flow-inducing element 582 can be changed by reversing the directionality of rotation of the driveshaft 310. In other cases, the directionality of the negative pressure created by the flow-inducing element 582 can be changed by reversing the angling of the one or more blades 584. In FIG. 5B, the one or more blades 584 of the flow-inducing element 582 are shown positioned in an opposite direction from the direction shown in FIG. 5A.

In at least some embodiments, a seal is disposed along the lumen 304 proximal to the inlet ports 592 and the outlet ports 594 to reduce the flow of the acoustically-favorable medium in a distal-to-proximal direction along the lumen 304. In at least some embodiments, the seal allows flow in a proximal-to-distal direction for normal flushing, but provides a higher resistance to flow in a distal-to-proximal direction, thereby allowing the fluid to exit the lumen 304 via the outlet ports 594.

The amount of the acoustically-favorable medium pumped through the lumen 304 may be affected by many different factors including, for example, the diameter, number, and location of the inlet ports 592 and outlet ports 594, the rotational velocity of the flow-inducing element 582, the size and shape of the blades 584, the amount of clearance between the blades 584 and inner walls of the lumen 304, or the like.

In at least some embodiments, the lumen 304 is at least partially filled with the acoustically-favorable medium prior to use. In at least some embodiments, the lumen 304 is at least partially filled with the acoustically-favorable medium prior to an imaging procedure such that the flow-inducing element 582 remains in contact with at least some of the acoustically-favorable medium.

Turning to FIGS. 6A-6B, in at least some embodiments the flow-inducing element may be used with an imaging core that includes a motor (see e.g., FIG. 4). In at least some embodiments, the motor is magnetic and includes a magnet configured and arranged to rotate by a magnetic field generated by a plurality of magnetic field windings. In at least some embodiments, the magnetic field windings are disposed at least partially in the imaging core. In other embodiments, the magnetic field windings are disposed external to the imaging core.

When the imaging core includes a rotating magnet, the amount of torque generated by the magnet may be related to the size of the magnet. In some cases, it may be advantageous to increase the size of the magnet in order to increase the amount of torque generated by the magnet. Increasing the size of the magnet, however, may decrease the number of blood vessels within which the catheter may be advanced, due to size constraints.

As herein described, the distal portion of the sheath includes a distal imaging region having a diameter that is smaller than a diameter of the remaining distal portion of the sheath proximal to the distal imaging region. The one or more transducers are extended away from the magnet, via the driveshaft. In at least some embodiments, the one or more transducers are extendable into the distal imaging region. In at least some embodiments, the magnet is configured and arranged to fit in the distal end of the catheter proximal to the distal imaging region, but is too large to fit in the distal imaging region.

In at least some embodiments, the catheter defines one or more inlet ports defined along the distal portion of the sheath. In at least some embodiments, the catheter defines one or more outlet ports defined along the distal portion of the sheath. In at least some embodiments, the flow-inducing element is disposed on the driveshaft and is configured and arranged to draw an acoustically-favorable medium into the lumen of the catheter, via the one or more inlet ports, and output the acoustically-favorable medium and gasses from the one or more outlet ports. In at least some embodiments, the flow-inducing element and the one or more transducers are disposed between at least one of the inlet ports on one end and at least one of the outlet ports on an opposing end such that the flow-inducing element induces flow of the acoustically-favorable medium across the one or more transducers.

FIG. 6A is a schematic longitudinal cross-section view of one embodiment of a distal portion 652 of a sheath 604 of a catheter 602 disposed in a blood vessel 662 with blood flow in a first direction. FIG. 6B is a schematic longitudinal cross-section view of one embodiment of the distal portion 652 of the sheath 604 disposed in the blood vessel 662 with blood flow in a second direction that is opposite from the first direction. The sheath 604 defines a lumen 606. An imaging core 608 is disposed in the lumen 606 of the sheath 604 at the distal portion 652 of the sheath 604.

The imaging core 608 includes a rotatable driveshaft 610 with an imaging device one or more transducers 612 coupled to a distal end of the driveshaft 610, and a transformer 614 coupled to a proximal end of the driveshaft 610. The imaging core 608 also includes a motor 616 coupled to the driveshaft 610. In at least some embodiments, the motor 616 includes a rotating magnet 626 and windings 628. In FIGS. 6A-6B, the windings 628 are shown fully disposed fully in the imaging core 608. In at least some embodiments, the windings 628 may be only partially disposed in the imaging core 608. In alternate embodiments, the windings 28 are disposed external to the imaging core 608.

The catheter 602 includes a distal imaging region 672 having a diameter 674 that is smaller than a diameter 676 of the catheter 602 at the portion of the distal end 652 of the catheter 602 proximal to the distal imaging region 672. The diameter 674 of the distal imaging region 672 may be smaller than the diameter 676 by at least 10%, 20%, 30%, 40%, 50%, 60%, 70%, or more. It may be advantageous to design the catheter 602 with the distal imaging region 672 having a smaller diameter than the portion of the catheter 602 immediately proximal to the distal imaging region 672 to increase the number of blood vessels that may be imaged using the imaging system by decreasing the diameter of the catheter at the location of imaging. It may further be advantageous to decrease the diameter of the catheter at the location of imaging without sacrificing torque due to a corresponding decrease in the size of the magnet 626.

The driveshaft 610 may have any suitable longitudinal length. In at least some embodiments, the driveshaft 610 is long enough to longitudinally separate the proximal-most portion of the one or more transducers 614 from the distal-most portion of the motor 616 by at least 2 cm, 3 cm, 4 cm, 5 cm, 6 cm, 7 cm, 8 cm, 9 cm, 10 cm, or more. In at least some embodiments, the driveshaft 610 is configured and arranged to extend the one or more transducers 612 into the distal imaging region 672. In at least some embodiments, the motor 616 is sized such that the motor 616 is prevented from extending into the distal imaging region 672. In at least some embodiments, the motor 616 has a diameter 678 that is larger than the diameter 674 of the distal imaging region 672.

A flow-inducing element 682 is disposed in the sheath 604 and is in fluid communication with the lumen 606 and the one or more transducers 612. In at least some embodiments, the flow-inducing element 682 is configured and arranged to rotate during an imaging procedure. In at least some embodiments, the flow-inducing element 682 is configured and arranged to rotate whenever the driveshaft 610 rotates.

In at least some embodiments, the flow-inducing element 682 enables air to be flushed from the lumen 606 and replaced by an acoustically-favorable medium disposed in the environment external to (and adjacent to) the catheter 602. Thus, when the catheter 602 is disposed in the blood vessel 662, blood may be pumped through the lumen 606 of the catheter 602. In at least some embodiments, the flow-inducing element 682 enables air to be flushed from the lumen 606 prior to an IVUS imaging procedure. In at least some embodiments, the flow-inducing element 682 enables air to be flushed from the lumen 606 during an IVUS imaging procedure.

In FIGS. 6A-6B, the flow-inducing element 682 is shown disposed on the driveshaft 610. In at least some embodiments, the flow-inducing element 682 is affixed (e.g., using an adhesive, interference fit, or the like) to the driveshaft 610. In at least some embodiments, the flow-inducing element 682 and the driveshaft 610 are inseparable from one another. In at least some embodiments, the flow-inducing element 682 and the driveshaft 610 are formed from a single piece of material (i.e., a unitary structure). In at least some embodiments, the flow-inducing element 682 is fixedly coupled to the driveshaft 610 such that rotation of the driveshaft 610 causes a corresponding rotation of the flow-inducing element 682.

In at least some embodiments, the flow-inducing element 682 is formed as one or more impellers, screw pumps, or the like or combinations thereof. The flow-inducing element 682 may include one or more blades that are either coupled directly to the driveshaft 610, or coupled to a sleeve that, in turn, is coupled to the driveshaft 610.

The sheath 604 defines one or more inlet ports, such as inlet port 692, and one or more outlet ports 694. The one or more inlet ports 692 and one or more outlet ports 694 enable fluid from the environment external to the sheath 604 to enter and exit, respectively, the lumen 606. In at least some embodiments, the flow-inducing element 682 is configured and arranged to push fluid and gas out of the lumen 606 through the one or more outlet ports 694, thereby creating a negative pressure within the lumen 606 that draws additional fluid into the lumen 606 from a location external to the sheath 604.

In at least some embodiment, the fluid is drawn along the lumen 606 such that the fluid flows over the one or more transducers 612. Thus, for example, when the catheter 602 is disposed in a blood vessel, such as the blood vessel 662, operation of the flow-inducing element 682 causes blood (which is typically an acoustically-favorable medium) to enter into the lumen 606 through at least one of the one or more inlet ports 692, flow along at least a portion of the lumen 606 over the one or more transducers 612, and exit the lumen 606 (along with gas) through the one or more outlet ports 694.

The sheath 604 may define any suitable number of inlet ports 692 including, for example, one, two, three, four, five, six, seven, eight, nine, ten, twenty, thirty, forty, fifty, or more inlet ports 692. The one or more inlet ports 692 can be formed in any suitable shape including, for example, linear, round, oval, triangular, rectangular, or the like or combinations thereof. The one or more inlet ports 692 can be any suitable non-geometric shape including, for example, cruciform, wavy line, or the like or combination s thereof. The one or more inlet ports 692 may also be irregularly-shaped. The one or more inlet ports 692 may be formed in the sheath 604 using any suitable technique (e.g., laser drilling, or the like).

The one or more inlet ports 692 may extend through the sheath 604 at any suitable angle relative to a longitudinal length of the catheter 602. In at least some embodiments, at least one of the one or more inlet ports 692 extends perpendicular to the longitudinal length of the catheter 602. In at least some embodiments, at least one of the one or more inlet ports 692 extends parallel to the longitudinal length of the catheter 602. In at least some embodiments, at least one of the one or more inlet ports 692 extends through the sheath 604 at an angle that is neither perpendicular nor parallel to the longitudinal length of the catheter 602 (e.g., at a slant).

In at least some embodiments, at least one of the one or more inlet ports 692 is slanted such that the inlet port 692 opens into the lumen 606 at a location that is more distal along the catheter 602 than an opposing opening of the inlet port 692 onto an outer surface of the sheath 604. In at least some embodiments, at least one of the one or more inlet ports 692 is slanted such that the inlet port 692 opens into the lumen 606 at a location that is more proximal along the catheter 602 than an opposing opening of the inlet port 692 onto an outer surface of the sheath 604. In at least some embodiments, at least one of the one or more inlet ports 692 are slanted in the direction of overall blood flow within the blood vessel within which the catheter 602 is disposed such that, when the blood enters the lumen 606, the blood is diverted from its direction outside the catheter 602 by no more than ninety, eighty, seventy, sixty, fifty, forty, or thirty degrees. In FIG. 6A, the direction of blood flow within the blood vessel 662 is shown by a plurality of arrows, such as arrow 696.

The one or more inlet ports 692 may be defined at any suitable location along the distal portion 652 of the sheath 604. In at least some embodiments, at least one of the one or more inlet ports 692 is located proximal to the one or more transducers 612. In at least some embodiments, at least one of the one or more inlet ports 692 is located distal to the one or more transducers 612. In at least some embodiments, at least one of the one or more inlet ports 692 is located along the distal imaging region 672. In at least some embodiments, the one or more inlet ports 692 are disposed at a distal-most tip 632 of the catheter 602.

At least one of the one or more inlet ports 692 may be located proximal to the flow-inducing element 682, or distal to the flow-inducing element 682, or both. In embodiments where the driveshaft 610 is longitudinally translatable along the longitudinal length of the catheter 602, it may be advantageous to longitudinally-offset at least one of the inlet ports 692 from at least one of the outlet ports 694 along the longitudinal length of the catheter 602 so that at least one of the inlet ports 692 is located on an opposing side of the flow-inducing element 682 from at least one of the outlet ports 692 regardless of the positioning of the flow-inducing element 682 at the distal end 652 of the catheter 602.

The one or more outlet ports 694 may be disposed at any suitable location along the distal portion 652 of the catheter 602. In at least some embodiments, at least one of the one or more outlet ports 694 is located distal to the one or more transducers 612. In at least some embodiments, at least one of the one or more outlet ports 694 is located proximal to the one or more transducers 612. In at least some embodiments, at least one of the one or more outlet ports 694 is located along the distal imaging region 672. In at least some embodiments, at least one of the one or more outlet ports 694 is disposed distal to at least one of the one or more inlet ports 692. In at least some embodiments, at least one of the one or more outlet ports 694 is disposed proximal to at least one of the one or more inlet ports 692. In at least some embodiments, each of the one or more outlet ports 694 is disposed distal to each of the one or more inlet ports 692. In at least some embodiments, each of the one or more outlet ports 694 is disposed proximal to each of the one or more inlet ports 692. In at least some embodiments, the one or more outlet ports 694 are disposed at a distal-most tip 632 of the catheter 602.

The sheath 604 may define any suitable number of outlet ports 694 including, for example, one, two, three, four, five, six, seven, eight, nine, ten, twenty, thirty, forty, fifty, or more outlet ports 694. The one or more outlet ports 694 can be formed in any suitable shape including, for example, linear, round, oval, triangular, rectangular, or the like or combinations thereof. The one or more outlet ports 694 can be any suitable non-geometric shape including, for example, cruciform, wavy line, or the like or combination s thereof. The one or more outlet ports 694 may also be irregularly-shaped. The one or more outlet ports 694 may be formed in the sheath 604 using any suitable technique (e.g., laser drilling, or the like).

The one or more outlet ports 694 may extend through the sheath 604 at any suitable angle relative to a longitudinal length of the catheter 602. In at least some embodiments, at least one of the one or more outlet ports 594 extends parallel to the longitudinal length of the catheter 602. In at least some embodiments, at least one of the one or more outlet ports 594 extends perpendicular to the longitudinal length of the catheter 602. In at least some embodiments, at least one of the one or more outlet ports 594 extends through the sheath 604 at an angle that is neither parallel nor perpendicular to the longitudinal length of the catheter 602 (e.g., at a slant). In at least some embodiments, at least one of the one or more inlet ports 692 is slanted such that the inlet port 692 opens into the lumen 606 at a location that is more distal along the catheter 602 than a location where that inlet port 692 opens to an outer surface of the sheath 604. In at least some embodiments, at least one of the one or more inlet ports 692 is slanted such that the inlet port 692 opens into the lumen 606 at a location that is more proximal along the catheter 602 than a location where that inlet port 692 opens to an outer surface of the sheath 604.

It may be advantageous to dispose at least one of the one or more inlet ports 692 at a location along the length of the catheter 602 such that, when acoustically-favorable medium (e.g., blood) external to the catheter 602 is drawn into the lumen 606 the acoustically-favorable medium passes over the one or more transducers 612 before exiting through the one or more outlet ports 694, thereby ensuring that the one or more transducers 612 remain immersed in the acoustically-favorable medium, and that the newly drawn acoustically-favorable medium (without gas) replaces the old acoustically-favorable medium (with gas).

It may be advantageous to position the one or more inlet ports 692 relative to the one or more outlet ports 694 such that the acoustically-favorable medium flows within the lumen 606 in the same direction as the overall flow of the acoustically-favorable medium in the blood vessel 662 external to the catheter 602, as shown by arrows 596 in FIG. 6A. Thus, in instances where the overall direction of blood flow within the blood vessel 662 is opposite from the directions indicated by the arrows 696 in FIG. 6A, it may be advantageous to reverse the positioning of the one or more inlet ports 592, and the one or more outlet ports 594.

FIG. 6B shows blood flowing in a direction indicated by arrows, such as arrow 698. The arrows 698 indicate a direction that is opposite from the direction shown by arrows 696 in FIG. 6A. Such a direction of flow may be present, for example, when imaging within a peripheral venous vessel. In FIG. 6B, the one or more inlet ports 692 and the one or more outlet ports 694 are positioned in opposite locations from the positions shown in FIG. 5A. In some cases, the directionality of the negative pressure created by the flow-inducing element 582 can be changed by reversing the directionality of rotation of the driveshaft 310. In other cases, the directionality of the negative pressure created by the flow-inducing element 582 can be changed by reversing the angling of the one or more blades, as described above with reference to FIGS. 5A-5B.

The amount of the acoustically-favorable medium pumped through the lumen 606 may be affected by many different factors including, for example, the diameter, number, and location of the inlet ports 692 and outlet ports 694, the rotational velocity of the flow-inducing element 682, the size and shape of the blades, the amount of clearance between the blades and inner walls of the lumen 606, or the like.

In at least some embodiments, the lumen 606 is at least partially filled with the acoustically-favorable medium prior to use. In at least some embodiments, the lumen 606 is at least partially filled with the acoustically-favorable medium prior to an imaging procedure such that the flow-inducing element 682 remains in contact with at least some of the acoustically-favorable medium.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. A catheter assembly for an ultrasound system, the catheter assembly comprising:
an elongated catheter configured and arranged for insertion into the cardiovascular system of a patient, the catheter having a distal end, a proximal end, and a longitudinal length, the catheter comprising a sheath with a proximal portion and a distal portion, the sheath defining a lumen extending along the sheath from the proximal portion to the distal portion;
an imaging core configured and arranged for inserting into the lumen of the catheter, the imaging core comprising
an elongated, rotatable driveshaft having a proximal end and a distal end, and
an imaging device coupled to the distal end of the driveshaft with rotation of the driveshaft causing a corresponding rotation of the imaging device, the imaging device comprising at least one transducer mounted to the imaging device, the at least one transducer configured and arranged for transforming applied electrical signals to acoustic signals and also for transforming received echo signals to electrical signals;
at least one inlet port defined in the distal portion of the sheath, the at least one inlet port extending between the lumen and the environment external to the catheter;
at least one outlet port defined in the distal portion of the sheath, the at least one outlet port extending between the lumen and the environment external to the catheter; and
a flow-inducing element coupled to the driveshaft with rotation of the driveshaft causing a corresponding rotation of the flow-inducing element, wherein the flow-inducing element is configured and arranged to draw fluid into the lumen through the at least one inlet port from the environment external to the sheath and push the drawn fluid out of the lumen through the at least one outlet port to the environment external to the sheath;
wherein one of the at least one inlet port or the at least one outlet port is disposed proximal to the flow-inducing element and the at least one transducer, and the other of the at least one inlet port or the at least one outlet port is disposed distal to the flow-inducing element and the at least one transducer.

2. The catheter assembly of claim 1, wherein the flow-inducing element comprises a screw pump.

3. The catheter assembly of claim 1, wherein the flow-inducing element comprises an impellor.

4. The catheter assembly of claim 1, wherein when the catheter is disposed in a patient blood vessel, the flow-inducing element is configured and arranged to draw blood from the blood vessel into the lumen through the at least one inlet port and push the drawn blood out of the lumen through the at least one outlet port to the blood vessel.

5. The catheter assembly of claim 1, wherein at least one of the at least one inlet ports is disposed proximal to the at least one transducer.

6. The catheter assembly of claim 1, wherein at least one of the at least one inlet ports is disposed distal to the at least one transducer.

7. The catheter assembly of claim 1, wherein at least one of the at least one inlet ports is disposed proximal to the at least one transducer and at least one of the at least one inlet ports is disposed distal to the at least one transducer.

8. The catheter assembly of claim 1, wherein at least one of the at least one inlet ports opens to the lumen at a location that is more distal than a location where that at least one inlet port opens to the environment external to the catheter.

9. The catheter assembly of claim 1, wherein the sheath has a distal-most tip, and wherein at least one of the at least one outlet ports is disposed at the distal-most tip.

10. The catheter assembly of claim 1, further comprising a drive unit coupled to the driveshaft, the drive unit configured and arranged for controlling rotation of the driveshaft.

11. The catheter assembly of claim 1, further comprising a control module coupled to the drive unit, the control module comprising
a pulse generator electrically coupled to the imaging core, the pulse generator configured and arranged for providing electric signals to the at least one transducer, and
a processor electrically coupled to the imaging core, the processor configured and arranged for processing received electrical signals from the at least one transducer to form at least one image.

12. A method of inducing flow of an acoustically-favorable medium over one or more transducers disposed in a lumen of a catheter of an ultrasound imaging system, the method comprising:
advancing the distal end of the catheter of claim 1 into a blood vessel of a patient, the blood vessel filled with flowing blood;
inserting the imaging core into the lumen of the sheath; and
rotating the driveshaft, the rotation of the driveshaft causing a corresponding rotation of the flow-inducing element and the at least one transducer, wherein rotation of the flow-inducing element causes blood from the blood vessel to enter the lumen through the at least one inlet port, flow over the at least one transducer, and flow out of the at least one outlet port, wherein the blood flowing within the lumen immerses the at least one transducer in the blood and flushes gas within the lumen out of the lumen through the at least one outlet port.

13. The method of claim 12, wherein the flow-inducing element comprises a screw pump.

14. A catheter assembly for an ultrasound system, the catheter assembly comprising:
an elongated catheter configured and arranged for insertion into the cardiovascular system of a patient, the catheter having a distal end with a first diameter, a proximal end, and a longitudinal length, the catheter comprising a sheath with a proximal portion and a distal portion, the sheath defining a lumen extending along the sheath from the proximal portion to the distal portion;
an imaging core with a longitudinal length that is substantially less than the longitudinal length of the catheter, the imaging core configured and arranged for inserting into the lumen of the catheter and disposing at the distal end of the catheter, the imaging core comprising
a rotatable driveshaft having a proximal end and a distal end,
an imaging device coupled to the distal end of the driveshaft, the imaging device comprising at least one transducer mounted to the imaging device with rotation of the driveshaft causing corresponding rotation of the imaging device, the at least one transducer configured and arranged for transforming applied electrical signals to acoustic signals and also for transforming received echo signals to electrical signals,
a transformer disposed at the proximal end of the driveshaft,
at least one imaging core conductor coupling the at least one transducer to the transformer, and
a motor having a diameter, the motor coupled to the driveshaft between the at least one transducer and the transformer, the motor configured and arranged to rotate the driveshaft, the motor comprising a magnet driven to rotate by at least two magnetic field windings;
at least one inlet port defined in the distal portion of the sheath, the at least one inlet port extending between the lumen and the environment external to the catheter;
at least one outlet port defined in the distal portion of the sheath, the at least one outlet port extending between the lumen and the environment external to the catheter; and
a flow-inducing element coupled to the driveshaft with rotation of the driveshaft causing a corresponding rotation of the flow-inducing element, wherein the flow-inducing element is configured and arranged to draw fluid into the lumen through the at least one inlet port from the environment external to the sheath and push the drawn fluid out of the lumen through the at least one outlet port to the environment external to the sheath;
wherein one of the at least one inlet port or the at least one outlet port is disposed proximal to the flow-inducing element and the at least one transducer, and the other of the at least one inlet port or the at least one outlet port is disposed distal to the flow-inducing element and the at least one transducer.

15. The flow-inducing element of claim 14, wherein the distal end of the catheter comprises a distal imaging region, and wherein distal imaging region has a second diameter that is smaller than the first diameter.

16. The flow-inducing element of claim 14, wherein the distal imaging region is configured and arranged to receive the distal end of the driveshaft and the at least one transducer.

17. The flow-inducing element of claim 14, wherein the diameter of the motor is larger than the second diameter.

18. The catheter assembly of claim 14, wherein the flow-inducing element comprises at least one of a screw pump or an impellor.

19. The catheter assembly of claim 14, wherein when the catheter is disposed in a patient blood vessel, the flow-inducing element is configured and arranged to draw blood from the blood vessel into the lumen through the at least one inlet port and push the drawn blood out of the lumen through the at least one outlet port to the blood vessel.

20. A method of inducing flow of an acoustically-favorable medium over one or more transducers disposed in a lumen of a catheter of an ultrasound imaging system, the method comprising:
advancing the distal end of the catheter of claim 14 into a blood vessel of a patient, the blood vessel filled with flowing blood;
inserting the imaging core into the lumen of the sheath; and
rotating the driveshaft, the rotation of the driveshaft causing a corresponding rotation of the flow-inducing element and the at least one transducer, wherein rotation of the flow-inducing element causes blood from the blood vessel to enter the lumen through the at least one inlet port, flow over the at least one transducer, and flow out of the at least one outlet port, wherein the blood flowing within the lumen immerses the at least one transducer in the blood and flushes gas within the lumen out of the lumen through the at least one outlet port.
